(19) United States
(12) Patent Application Publication
(10) Pub. No.: US 2025/0322919 A1
(43) Pub. Date: Oct. 16, 2025

(54) SYSTEM AND METHOD FOR THE DESIGN AND MANAGEMENT OF CLINICAL TRIALS

(71) Applicants: Christopher Wrenn Herlong, Franklin, TN (US); Temple Shea Herlong, Franklin, TN (US)

(72) Inventors: Christopher Wrenn Herlong, Franklin, TN (US); Temple Shea Herlong, Franklin, TN (US)

(21) Appl. No.: 19/098,419

(22) Filed: Apr. 2, 2025

Related U.S. Application Data
(60) Provisional application No. 63/633,385, filed on Apr. 12, 2024.

(51) Int. Cl. G16H 10/20; G16H 10/60

(52) U.S. Cl. CPC G16H 10/20; G16H 10/60

(57) ABSTRACT

A system and method for clinical trial design and management using an automated and integrated architecture. Clinical trial requirements are identified and used for automatically generating a clinical trial design and clinical trial timeline, a clinical trial protocol, one or more informed consents, one or more custom report forms (CRFs), and one or more electronic data control (EDC) specifications. The trial design and clinical trial timeline, the clinical trial protocol, the one or more informed consents, the one or more CRFs, and the one or more EDC specifications are generated using a common workflow. All this is generated while simultaneously assembling designs pre-populated with a standard trial architecture and required data points, validating configuration, cross-checking enrollment numbers and study timelines, populating the protocol with supporting data and visuals, and generating a customized technical specifications document for use with an existing clinical EDC system.

### CROSS-REFERENCE TO RELATED APPLICATION

This application claims the benefit of U.S. Provisional Patent Application No. 63/633,385 filed Apr. 12, 2024, which is hereby incorporated by reference herein in its entirety.

### FIELD OF THE INVENTION

The present invention relates generally to clinical trials, and more particularly to a system and method for designing and managing clinical trials in a fully integrated architecture.

### BACKGROUND OF THE INVENTION

In the United States, before a new medical drug (e.g., pharmaceuticals) or device (e.g., surgical instruments or implants) may be dispensed to the public, the United States Food and Drug Administration (FDA) requires that the manufacturers of the pharmaceuticals, devices, instruments, or implants conduct extensive clinical trial research in order to demonstrate the clinical effectiveness, safety, and medical advantage of their products. In particular, obtaining approval for a therapeutic product (e.g., a medical device, a pharmaceutical product such as a drug, etc.) requires a clinical trial in which the therapeutic product is tested on human subjects to validate the product's safety and efficacy for its intended purpose. To ensure that the results of the clinical trials are reliable and that results are reproducible, many clinical trials are often multi-site and/or multinational operations which typically require substantial planning and oversight to run efficiently. Extensive and often complex clinical trial protocols are developed that define, for example, targeted demographics, proposed medications, patient regimens, forms for collection, types of statistically relevant data, the timing or order of events within the study, often even the layout of the reporting data, and the like. These protocols are often sufficiently complex so that the protocols themselves receive FDA approval. For example, a clinical trial may involve hundreds or thousands of patients recruited worldwide, and a central management service may be employed to manage various aspects of the clinical trial.

The National Institute of Health (NIH) defines a clinical trial as a research study in which one or more human subjects are prospectively assigned to one or more interventions (which may include placebo or other control) to evaluate the effects of those interventions on health-related biomedical or behavioral outcomes. When a research institute, medical facility, medical device company, or pharmaceutical company has a new idea for the improved treatment or management of a disease, they must first perform a series of clinical trials and submit their data for approval by the FDA prior to releasing it to the public. The design, planning, and execution of these critical trials and the FDA approval process is an expensive undertaking and may take years from start to finish.

Critical to today's clinical trial design efforts is an Electronic Data Capture (EDC) system which is a software product that is utilized by life sciences organizations who perform research studies in a regulated environment. There are many EDC products commercially available, and each product is used to record and manage the research data that is generated for the purposes of a clinical research study. Current commercially available EDC systems include, but are not limited to, Castor EDC, ClinCapture, ClinInfo eCRF, Fountayn/Datatrak, DSG eCaseLink, Ennov EDC, IBM/Merative CD, iMedNet, Medrio, Medidata RAVE, REDCap Cloud, TrialKit, and Veeva CDMS. Typical EDC products have standard data forms that can be used for any study, workflow rules to help with data entry, logic checks to verify the quality of the data and reporting metrics for progress. In general, EDC products are used to record specific data about individual subjects (e.g., patients) that participate in research studies. For example, if a biopharmaceutical organization is testing their new diabetes drug in 200 subjects at 10 medical centers, each medical center will use the EDC to enter the research data about their participating study subjects. Before the medical centers are able to record the research data in the EDC, the data forms have to be designed with the appropriate pages and fields that relate to the diabetes research study. Typically, the sponsor organization will work with the EDC vendor or provider to design the pages and fields needed for their research study. Once the EDC is ready to use, the sponsor organization or their representatives will train the research site users how to use the EDC for the specific study. Because each EDC system is unique and each research study is unique, knowing how to navigate through the systems is critical for quality research data. In general, data that is entered into an EDC includes a combination of personal health information (PHI) as well as general information about each subject participating in the research study. When a subject signs informed consent to participate in a study, the details of the study are provided. Most studies will collect some background information about each participant. For example, the person's age, recent bloodwork, whether they have any ongoing medical conditions and details about the research study disease (e.g., severity of their diabetes condition, what medications they are taking, etc.). Study-specific information, also known as “research data”, will also be collected and recorded during the study. For example, each time the subject comes to the clinic for a site visit, they may be asked to complete a survey about how they are feeling, provide details about the time they take their medication each day and have a brief physical exam. These items are “research data” because the patient would not normally be reporting this detail to their doctor for routine diabetes care. Additional information may be collected in other systems such as laboratories and imaging facilities. For example, cancer studies may require repeat scans and bloodwork. Some EDC systems can “connect” with these additional systems. In general, subject data entered into an EDC system does not contain information to identify subjects (e.g., the person's full name, address, contact details).

Thus. in order to facilitate the development and approval for protocols and their related clinical trials companies employ various electronic data capture and data management solutions to manage the massive amount of data gathered. In general, such data capture and data management solutions capture data from geographically disparate clinicians or study participants defining many points of data entry, potentially across multiple software and hardware platforms. Drawbacks of these systems include the costs associated with designing, customizing, and/or adapting particular proprietary data capture and data management systems to the ever changing needs presented with each new protocol of each new clinical trial. The design process itself is labor intensive and lengthy. Thus, many of today's clinical trial sponsors employ highly-skilled technology personnel, such as computer programmers or system designers, to adapt their systems, particularly their database and data storage systems, to each protocol. Use of such personnel can be time-consuming, costly, inefficient, and can lack scalability.

Accordingly, there is a need for improving and integrating clinical trial design and management.

### SUMMARY OF THE INVENTION

The present invention is directed to a system and method for clinical trial design and management using an automated and integrated architecture.

In a first implementation of the invention, a trial design and management system for clinical trial design and management using an automated and integrated architecture. The trial design and management system comprising at least: a processor, and a memory storing instructions that when executed cause the processor to perform operations comprising: (i) receiving a plurality of clinical trial requirements specific to a targeted clinical trial having a clinical research type and a trial type; (ii) generating from the plurality of clinical trial requirements received (a) a clinical trial design and clinical trial timeline; (b) a clinical trial protocol; (c) one or more informed consents; (d) one or more case report forms (CRFs); and (e) one or more electronic data capture (EDC) specifications; (iii) transmitting the trial design and clinical trial timeline generated, the clinical trial protocol generated, one or more informed consents generated, the one or more case report forms generated, and the one or more electronic data capture (EDC) specifications generated to an EDC system; (iv) receiving from the EDC system a clinical trial database design specific to collecting data for the targeted clinical trial in accordance with the trial design and clinical trial timeline generated, the clinical trial protocol generated, the one or more informed consents generated, the one or more case report forms generated, and the one or more EDC specifications generated; and (v) validating the clinical trial database design received using the one or more EDC specifications generated.

In a second aspect, a method is provided for clinical trial design and management using an automated and integrated architecture. The method comprising: (i) receiving a plurality of clinical trial requirements specific to a targeted clinical trial having a clinical research type and a trial type; (ii) generating from the plurality of clinical trial requirements received (a) a clinical trial design and clinical trial timeline; (b) a clinical trial protocol; (c) one or more informed consents; (d) one or more case report forms; and (e) one or more electronic data capture (EDC) specifications; (iii) transmitting the trial design and clinical trial timeline generated, the clinical trial protocol generated, the one or more informed consents generated, the one or more case report forms generated, and the one or more electronic data capture (EDC) specifications generated to an EDC system; (iv) receiving from the EDC system a clinical trial database design specific to collecting data for the targeted clinical trial in accordance with the trial design and clinical trial timeline generated, the clinical trial protocol generated, the one or more informed consents generated, the one or more case report forms generated, and the one or more EDC specifications generated; and (v) validating the clinical trial database design received using the one or more EDC specifications generated.

In a third aspect, a method is provided for clinical trial design and management using an automated and integrated architecture that eliminates the need for using any EDC system. The method comprising: (i) receiving a plurality of clinical trial requirements specific to a targeted clinical trial having a clinical research type and a trial type; (ii) generating from the plurality of clinical trial requirements received (a) a clinical trial design and clinical trial timeline; (b) a clinical trial protocol; (c) one or more informed consents; (d) one or more case report forms; and (e) one or more electronic data capture (EDC) specifications; (iii) presenting the clinical trial design and clinical trial timeline, clinical trial protocol, one or more informed consents, and one or more case report forms for approval, (iv) responsive to receiving the approval, generating a clinical trial database design specific to collecting data for the targeted clinical trial in accordance with the trial design and clinical trial timeline generated, the clinical trial protocol generated, the one or more informed consents generated, the one or more case report forms generated, and the one or more EDC specifications generated; and (v) validating the clinical trial database design generated; (vi) managing data collection through execution of the targeted clinical trial in accordance with the clinical trial design and clinical trial timeline; and (vii) analyzing the data collected through execution of the targeted clinical trial in accordance with the clinical trial design and clinical trial timeline and generating one or more reports.

In a fourth aspect, a trial designer application (alternatively referred to herein as an “app”) may be executed on the trial design and management system and/or another data processing system for executing at least the method operations for clinical trial design and management using an automated and integrated architecture.

In a fifth aspect, a trial management application may be executed on the trial design and management system and/or another data processing system for clinical trial design and management using an automated and integrated architecture that eliminates the need for using any EDC system.

In another aspect, one or more location-specific clinical research participant consent documents are generated.

In another aspect, the validated clinical trial database design is presented for user approval.

In another aspect, the clinical research type and the trial type are recommended by the trial design and management system based on the targeted clinical trial.

In another aspect, the trial design and clinical trial timeline, the clinical trial protocol, the one or more informed consents, the one or more case report forms, and the one or more electronic data capture (EDC) specifications are generated using a common workflow.

In another aspect, there is a correlating the clinical trial database design received, the trial design and clinical trial timeline generated, the clinical trial protocol generated, the one or more informed consents generated, the one or more case report forms generated, and the one or more electronic data capture (EDC) specifications generated with one or more standards promulgated by a Clinical Data Interchange Standards Consortium (CDISC).

In another aspect, a library of common calculations associated with the targeted clinical trial are accessed.

In another aspect, there is a nesting multiple ones of the common calculations from the library of common calculations in at least one case report from the one or more case reports generated.

In another aspect, the clinical trial design and clinical trial timeline generated provide for clinical trial data collection on a per site level and a per study level.

In another aspect, one or more groups and one or more crossovers are defined and incorporated into at least the clinical trial timeline generated.

In another aspect, the one or more groups defined further comprise a plurality of categories defined by phases, parts, cohorts, arms, and crossovers.

In another aspect, each one of the phases, parts, cohorts, arms, and crossovers require a start date, duration, enrollment goal, and enrollment limit.

In another aspect, the one or more crossovers defined further comprise a crossover type, related arms, crossover direction, at least two potential start dates, subject eligibility, and maximum number of subjects allowed to crossover.

In another aspect, a plurality of visits are mapped for which a plurality of subjects are to attend in accordance with the clinical trial design and clinical trial timeline generated.

In another aspect, a plurality of forms, surveys, and/or electronic patient reported outcomes (ePRO) are mapped with the plurality of visits in accordance with the clinical trial design and clinical timeline generated.

In another aspect, a plurality of data collection points are mapped with the plurality of forms in accordance with the clinical trial design and clinical timeline generated.

In another aspect, there is a correlating the plurality of data collection points with pre-defined naming conventions promulgated by a Clinical Data Interchange Standards Consortium (CDISC).

The methods and systems described herein can be implemented by data processing systems, such as one or more smartphones, tablet computers, desktop computers, laptop computers, smart watches, wearable, audio accessories, on-board computer, and other user devices and consumer electronic devices. The methods and systems described herein can also be implemented by one or more data processing systems which execute executable computer program instructions, stored in one or more non-transitory machine readable media that cause the one or more data processing systems to perform the one or more methods described herein when the program instructions are executed. Thus, the embodiments described herein can include methods, data processing systems, and non-transitory machine readable media.

The above summary does not include an exhaustive list of all embodiments in this disclosure. All systems and methods can be practiced from all suitable combinations of the various aspects and embodiments summarized above, and also those disclosed in the detailed description below.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a diagram showing the conventional phases of a clinical trial;

FIG. 2 presents a flowchart of illustrative operations for a conventional clinical trial design and management process;

FIG. 3 presents a flowchart of illustrative operations for a client trial design process in accordance with an embodiment;

FIG. 4 presents a clinical trial design process architecture and workflow incorporating the client design process of FIG. 3 in accordance with an embodiment;

FIG. 5 presents a holistic clinical trial design and management process architecture and workflow that eliminates the need for an EDC system in accordance with an embodiment;

FIG. 6 presents illustrative architectures for a trial designer app and a trial design and management app, respectively, in accordance with an embodiment;

FIG. 7 presents a high-level block diagram of a cloud network services architecture for providing client trial design and management in accordance with an embodiment;

FIG. 8 presents an illustrative a trial design and management system configured for providing clinical trial design and management in accordance with an embodiment;

FIG. 9 presents a user device configured for providing clinical trial design and management in accordance with an embodiment;

FIG. 10 presents a high-level operational overlay diagram of the trial design and workflow architecture of FIG. 4 with the illustrative architectures for the trial designer app of FIG. 6 in accordance with an embodiment;

FIGS. 11 and 12 present illustrative outputs from executing the trial design app of FIG. 6 employing the trial design and workflow architecture of FIG. 10 in accordance with an embodiment; and

FIG. 13 presents a high-level visualization of the clinical trial design process architecture and workflows herein as compared to traditional systems in accordance with an embodiment.

Like reference numerals refer to like parts throughout the several views of the drawings.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed toward a system and method for clinical trial design and management using an automated and integrated architecture. That is, in accordance with the principles of the disclosed embodiments, the typical complex inner workings of clinical trial design are simplified and automated for providing improved clinical design and ensuring FDA and CDISC compliance, and dramatically reducing development timelines and costs. Significantly, the principles of the disclosed embodiments starts the clinical design process by identifying and gathering clinical trial requirements which is in contrast to conventional clinical trial design processes that begin with a manually developed protocol. In this way, the system and method herein use the identified and gathered clinical trial requirements for automatically generating a clinical trial design and clinical trial timeline, a clinical trial protocol, one or more informed consents, one or more CRFs, and one or more EDC specifications. All this is generated while simultaneously assembling designs pre-populated with a standard trial architecture and required data points, validating configuration, cross-checking enrollment numbers and study timelines, populating the protocol with supporting data and visuals, and generating a customized technical specifications document for use with an existing clinical EDC system. In a further embodiment, the system and method are configured to completely eliminate the need and use of an EDC system in clinical design and management. As result, the clinical trial design and management system and method of the disclosed embodiments replace a traditionally error-prone, manual process that occurs between clinical trial conception and “go live” of the designed clinical trial in addition to providing an advantageous improvement of practical applications that include clinical trial design and management systems and EDC systems.

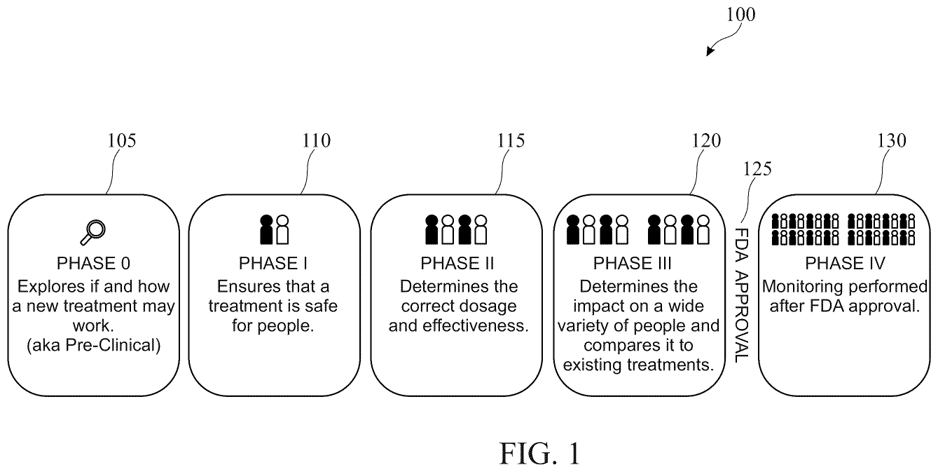

Turning our attention to FIG. 1, a diagram 100 showing the conventional phases of a clinical trial is presented. As will be appreciated by one of ordinary skill in the art, the National Institute of Health (NIH) defines a clinical trial as a research study in which one or more human subjects are prospectively assigned to one or more interventions (which may include placebo or other controls) to evaluate the effects of those interventions on health-related biomedical or behavioral outcomes. Whenever a research institute, medical facility, medical device company, or pharmaceutical company has a new idea for the improved treatment or management of a disease, they must first perform a series of clinical trials and submit their data for approval by the FDA prior to releasing it to the general public. As shown in FIG. 1, clinical trials are typically broken into six (6) phases: (i) Phase 0 (see block 105) for exploring if and how a new treatment may work (aka Pre-Clinical); (ii) Phase I (see block 110) for ensuring the new treatment is safe for humans; (iii) Phase II (see block 115) for determining the correct dosage and effectiveness; (iv) Phase III (see block 120) for determining the impact on a wide variety of humans and comparing the results to existing treatments; (v) FDA Approval (see block 125); and (vi) Phase IV (sec block 130) monitoring the commercially new treatment after FDA approval is granted.

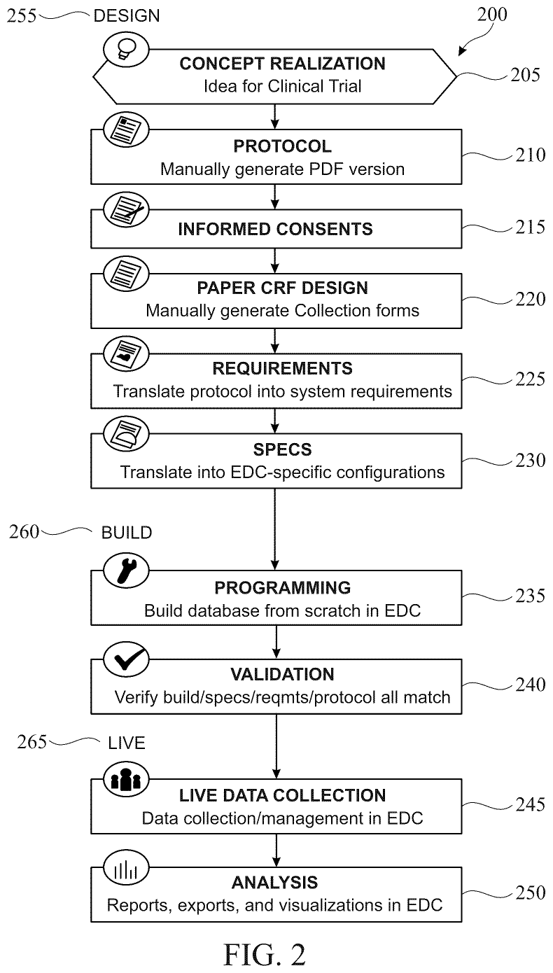

Turning our attention to FIG. 2, a flowchart of illustrative operations 200 for the aforementioned conventional clinical trial design and management process. More particularly, starting with a design phase 250 at block 205 there is identifying the concept realization for the clinical trial based on the proposed new treatment. At step 210, using the client trial conceptual realization a protocol is designed through a typical manual process. At step 215, one or more informed consents are received. Using the protocol design a series of data collections forms are also manually designed at block 220. Then, at block 225, the protocol design is translated to a set of system requirements which in turn are translated into a series of specifications directed to EDC-specific configurations based on the particular EDC system to be utilized. At block 230 and transitioning from the design phase 255 to a build phase 260, various programming is completed, at block 235, for building the requisite database using the EDC system that will be the repository of all the data collected during the clinical trial. Then, at block 240, a validation is performed for ensuring that there is suitable match between the clinical trial design, specifications, requirements, and protocol. Once validated and transitioning from the build phase 260 to a go live phase 265, the clinical trial is started thereby triggering various data collection and management operations, at block 245, using the EDC system. At block 250, using the data collected the EDC system then generates a variety of reports, exports, and visualizations for analysis purposes. Of course, the aforementioned conventional clinical trial design processes encompassing clinical trial development and management have been used successfully over a long period of time for introducing new FDA-approved treatments in the marketplace. That said, these clinical design processes and conducting formal clinical trials can be a very lengthy and costly endeavor. Further, these conventional processes introduce a high-degree of human error and rework possibilities given the reliance on various parties to perform a variety of manual tasks. For example, an error in the protocol generation (as noted above—block 210) that cascades through the subsequent procedures (i.e., blocks 215 through 240) requires a complete restart of the procedures back to protocol generation (i.e., block 210) in order to address the error and re-execute the subsequent procedures. As such, this conventional technique may be viewed as a “static” approach in that each procedure stands alone dependent on prior procedures but without any real-time or “dynamic” feedback loop. Addressing at least these type of issues with the conventional clinical trial design and management processes and significantly improving various parts thereof are the advancements and contribution made by the principles of the disclosed embodiments herein.

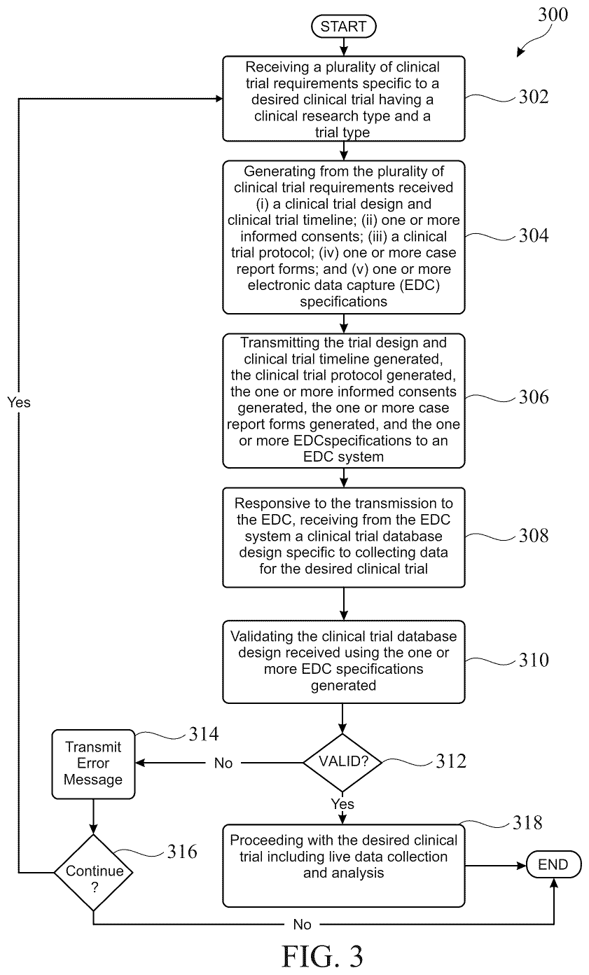

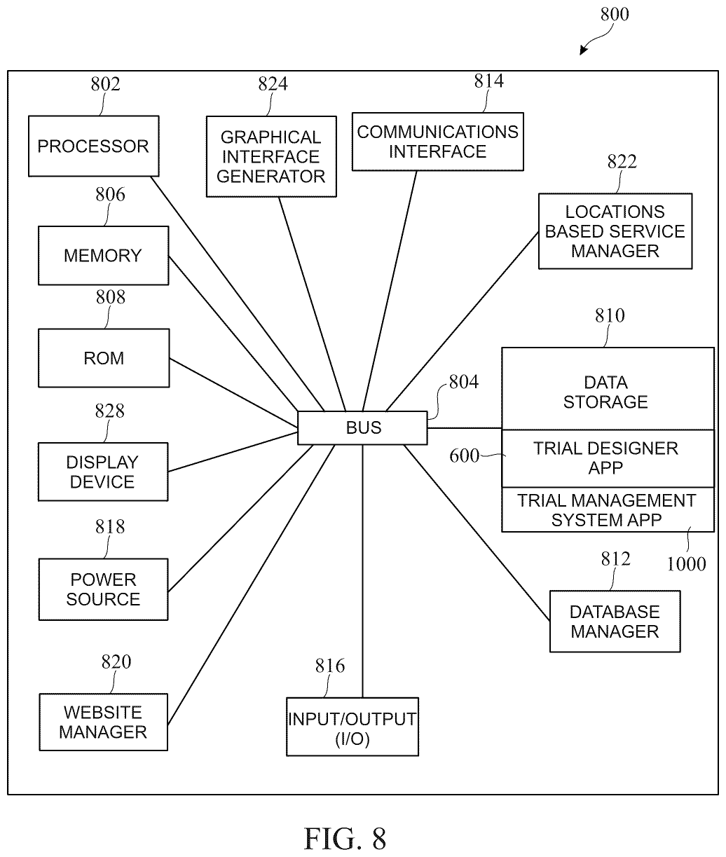

Turning our attention to FIG. 3, a flowchart of illustrative operations 300 is shown for a client trial design process in accordance with an embodiment. More particularly, at step 302, receiving a plurality of clinical trial requirements specific to a targeted clinical trial having a clinical research type and a trial type. Significantly, the principles of the disclosed embodiments starts the clinical design process by identifying and gathering clinical trial requirements which is inapposite to conventional clinical trial design processes that begin with a manually developed protocol, as detailed hereinabove. Advantageously, redefining and changing the procedural starting point from protocol generation to requirements identification thereby allows for an architecture that supports real-time or a “dynamic” feedback loop for automating the various procedures for on-the-fly corrections and adaptations leading to the automatic generation of the final clinical trial design and associated clinical trial timeline, clinical trial protocol, one or more informed consents, one or more case report forms, one or more EDC specifications as will be further detailed below. In this way, the principles of the disclosed embodiments eliminate the need to constantly restart the clinical trial design process due to error or oversights made in the protocol, for example, at the start. Further, each and every change made requires the requisite FDA approval to the extent the protocol has been previously approved but now requires changes due to the discovered error and/or oversights. This recognition of starting with the requirements and auto-generating the clinical trial protocol is a significant advancement made in terms of this disclosure and the various embodiments detailed. In an embodiment, based on the targeted clinical trial, a recommendation is made by the trial designer and management system 800 (see, FIG. 8) with respect to the appropriate clinical research type and the trial type for review and potential selection by the user.

In this way, at step 304, generating from the plurality of clinical trial requirements received (a) a clinical trial design and clinical trial timeline; (b) a clinical trial protocol; (c) one or more informed consents; (d) one or more case report forms; and (e) one or more electronic data capture (EDC) specifications, and transmitting, at step 306, the trial design and clinical trial timeline generated, the clinical trial protocol generated, the one or more informed consents generated, the one or more case report forms generated, and the one or more electronic EDC specifications generated to an EDC system. This demonstrates another significant advantage and advancement of the disclosed embodiments whereby the clinical trial design procedure now acts as a standalone tool that complements any existing EDC system and unlocks significant savings in terms of at least time and cost. Further, advantageously, the trial design and clinical trial timeline, the clinical trial protocol, the one or more informed consents, the one or more CRFs, and the one or more EDC specifications are generated using a common workflow. Also, in the generation thereof accessing a library of common calculations associated with the targeted clinical trial may be utilized and multiple ones of the common calculations from the library of common calculations may be nested in at least one of the CRFs generated. In an embodiment, correlating the clinical trial database design received, the trial design and clinical trial timeline generated, the clinical trial protocol generated, the one or more informed consents generated, the one or more CRFs generated, and the one or more EDC specifications generated with one or more standards promulgated by a Clinical Data Interchange Standards Consortium (CDISC). In an embodiment, one or more location-specific clinical research participant consent documents are also generated for use during the clinical trial. In the instant embodiment, after configuring the requirements, a technical specifications document customized to the EDC system of choice is generated and provided for use in building the clinical trial through that separate EDC system. In an embodiment, the clinical trial design generated provide for clinical trial data collection on a per site level and a per study level. In a further embodiment, detailed below, the clinical trial designer is fully integrated into the clinical EDC system and replaces the so-called “builder” in such EDC system thereby providing a single, user-guided, requirements-gathering workflow to concurrently build the clinical database, automate a significant amount of the advance programming needed, and eliminate the need for extensive validation.

At step 308, responsive to the transmission to the EDC system, receiving from the EDC system a clinical trial database design specific to collecting data for the targeted clinical trial in accordance with the trial design and clinical trial timeline generated, the clinical trial protocol generated, the one or more informed consents generated, the one or more case report forms generated, and the one or more EDC specifications generated. Then, at steps 310 and 312, validating the clinical trial database design received using the one or more EDC specifications generated. If not valid, transmitting an error message at step 314 and determining, at step 316, whether the user wants to continue with the clinical trial design procedures and if so, the operations return to step 302 and if not, the operations end. If the clinical trial database design is valid then proceeding, at step 318, to obtain user approval and if approved continuing with the desired clinical trial including, but not limited to, live data collection and analysis.

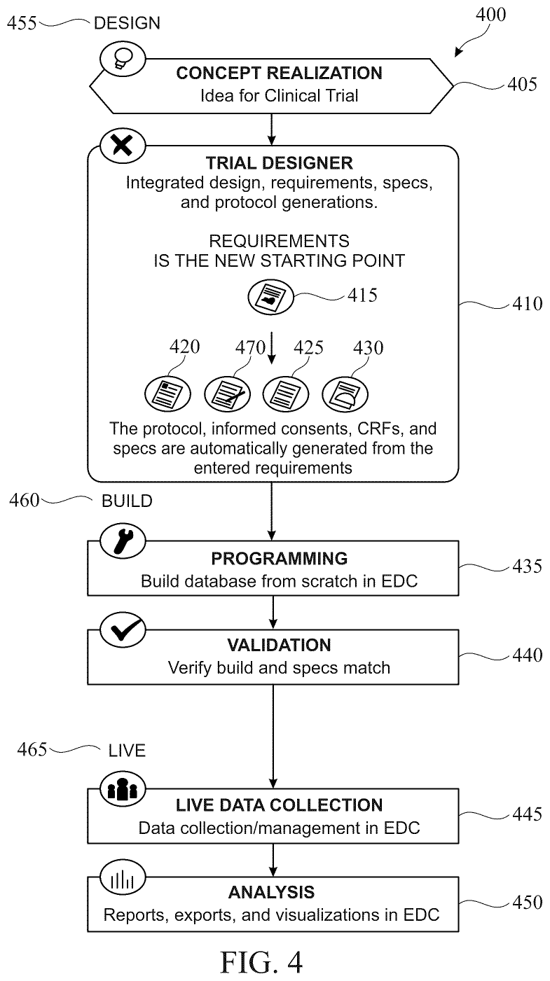

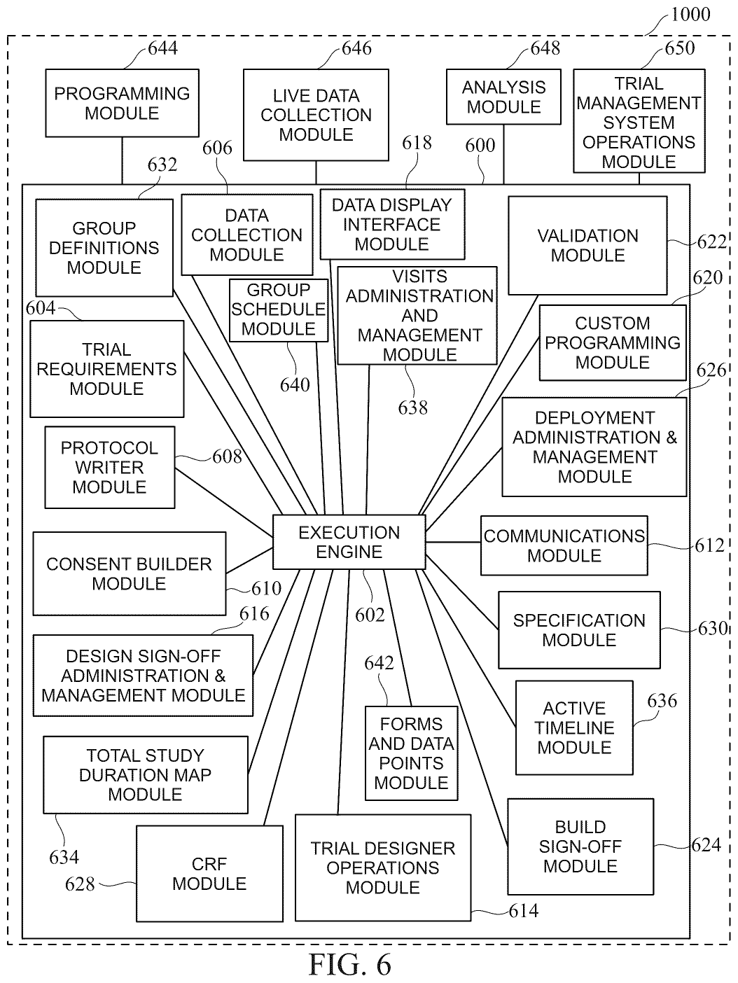

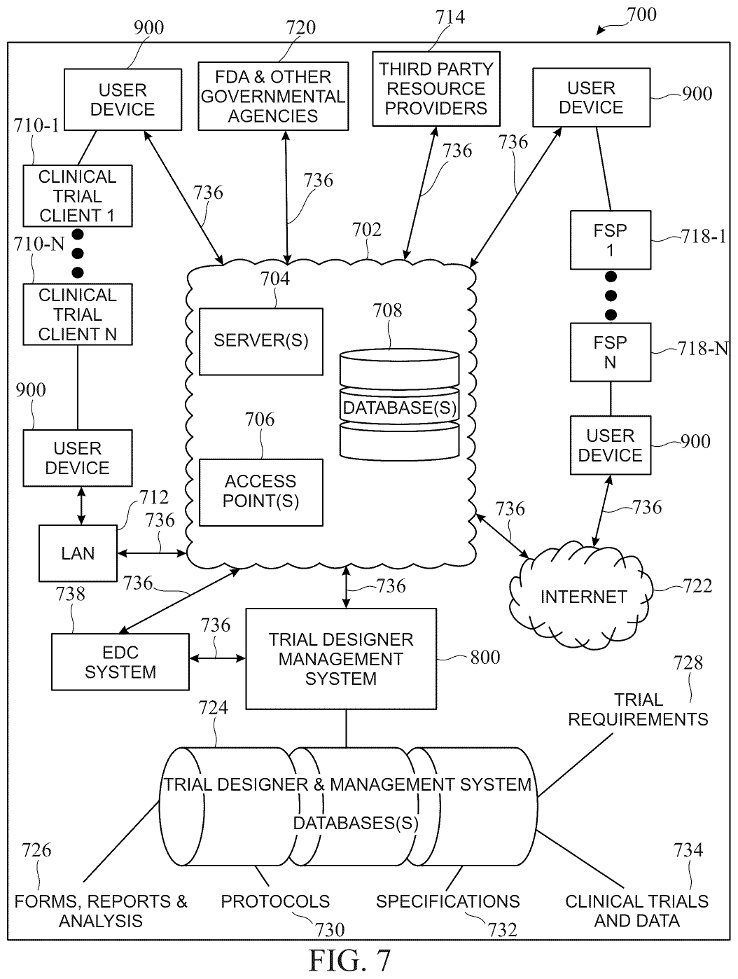

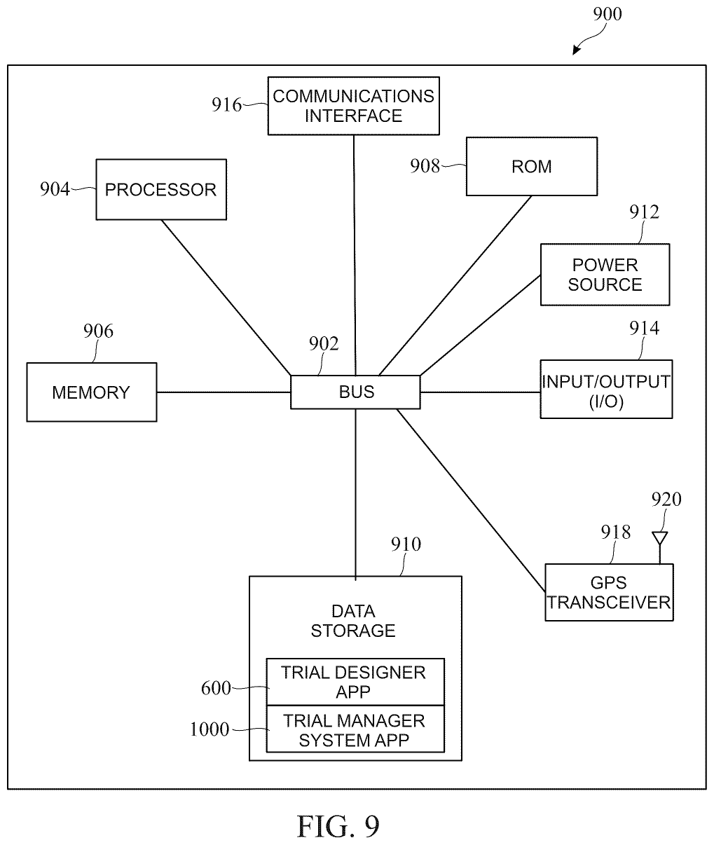

Turning our attention to FIG. 4, a clinical trial design process architecture and workflow 400 is shown for further advancing the understanding of the disclosed embodiments and incorporating at least the client design process of FIG. 3 in accordance with an embodiment. As will be detailed further hereinbelow, a trial design app 600 (sec, e.g., FIG. 6) is provided for execution on a trial designer and management system 800 (see, e.g., FIGS. 7 and 8) and/or a user device 900 (sec, e.g., FIGS. 7 and 9) in accordance with the principles of the disclosed embodiments. At block 405 and starting a design phase 455, the clinical trial idea and concept realization is made which includes selecting and identifying a clinical research type, a clinical trial type (dependent upon the clinical research type), and a trial design type (dependent upon the clinical trial type). Clinical research types include observational, diagnostic, interventional, and/or combinations thereof as defined by the NIH and FDA. Each clinical research type is associated with a particular set of clinical trial types as follows:

Design types structure a trial based on the selected research type and trial type. The designs deals primarily with the arrangement of phases, parts, cohorts, arms, groups, and crossovers. These terms all deal with the grouping and subgrouping of subjects to complete objective research. The clinical trial design types (as defined by the NIH) associated with the aforementioned clinical trial types are as follows:

The clinical trial design inclusive of the client research type, clinical trial type, and clinical design type form part of the requirements such that at block 410, the entirety of the requirements are identified at block 415. In accordance with the principles of the disclosed embodiments, the clinical trial requirements encompass at least the following:

### Trial Design

Research Type
Trial Type
Design Type

### Study Details

### Active Timeline

### Group Definitions

Phases
Parts
Cohorts
Arms
Crossovers

### Complete Timeline

### Visit Definitions and Schedules

Configuration
Trial Visits
Visit Schedule
Visit Cycles
Cycle Schedule

### Visit Timeline

### Group Visit Schedule

### Form Definitions

### Form Visit Schedule

### Data Points and Form Design

Data Points
Code Lists
Form Design

### Randomization and Blinding

### Clinical Supply Management

### Adjudication

### Form Visibility Schedule

### Form Accessibility Schedule

### Form Monitoring

### Advanced Programming

Custom Queries
Form Rules
Advanced Requirements

Significantly, certain of the above-identified trial requirements form a further part of the advancement and contribution made by the principles of the disclosed embodiments herein. That is, the active timeline (enrolled subject study participation map), group definitions, complete timeline (total study duration map), visit timeline, group visit schedule, form visibility schedule, and form accessibility schedule are features unique to the disclosed embodiments and are not found in any conventional EDC system. Illustratively, the active timeline is used for configuring a visual map that details the minimum and maximum active participation duration of subjects on the trial, based on data input into the system. It separately defines parts, cohorts, arms, and crossovers, along with individual durations and enrollment goals.

Typically, EDC software ignores “group” distinctions or simply defines it as a single entity. However, it is vital to break groups into five (5) subcategories consisting of Phases, Parts, Cohorts, Arms, and Crossovers. Each requires the defining of a start date, duration, enrollment goal, and enrollment limit, along with how they each relate to one another. The five different subcategories are created, configured, and managed easily through the previous Active Timeline step, but a separate step has been created to manage each group individually in a data grid design. As to phases, There are essentially five (5) study phases of clinical trial design: (1) Phase 0 explores if and how a new treatment may work; (2) Phase I ensures that the treatment is safe for people; (3) Phase II determines the correct dosage and effectiveness; (4) Phase III determines the impact on a wide variety of people and compares it to existing treatments and (5) Phase IV includes monitoring performed after FDA approval. In some cases, a single clinical trial will combine phases into a single timeline. A trial part is a smaller section of a larger trial, which has separately defined objectives, methods, and outcomes, but still plays an important part of the entire overall trial. Parts are typically in linear order and dependent on the preceding part. By default, trials will have one part assigned. A trial cohort is a group of subjects that share common, defining characteristics. By default, trials will have one cohort assigned. Arms are also referred to as treatment groups. An arm is a course of action that is applied to a group of subjects that differs from another course of action that is available. For instance, one arm may receive a new drug while a second arm may receive a placebo. Arms are typically managed in parallel and are not dependent on one another. Subjects may be manually assigned or randomized into an arm. Crossovers occur when an event or certain scenario allows for a subject to change from one arm to another. For instance, if a subject is randomized to an arm that is overly aggressive and resulting in adverse events, the trial may allow for the subject to crossover into a less aggressive arm so as to not lose that subject completely. However, some trials may have a scheduled event where subjects intentionally cross over to a different arm to further the research of the trial. Crossovers can be a planned event at a specified timepoint based on subject qualifications to extend or enhance the research. Crossovers can be unplanned, conditional events that may occur when a subject experiences an adverse event and needs to switch treatment for safety reasons. Crossovers are extremely complicated and significantly impact the timelines which is why conventional EDC software does not include this feature, but the principles of the disclosed embodiments successfully incorporate crossovers into the clinical trial design processes as another technological improvement and advancement over conventional systems. In particular, in an embodiment, one or more groups and one or more crossovers are defined which are then incorporated into at least the clinical trial timeline generated. Further, the one or more groups defined may further comprise a plurality of categories defined by phases, parts, cohorts, arms, and crossovers. Further, the one or more crossovers defined may further comprise a crossover type, related arms, crossover direction, at least two potential start dates, subject eligibility, and maximum number of subjects allowed to crossover.

The complete timeline requirement is direct to a visual map that details the minimum and maximum overall durations of the active trial, based on data input into the system. It separately defines the recruitment duration for an essential big picture snapshot. As to the visit definitions requirement, except for retrospective observational studies, clinical trials consist of a series of subject visits or events which can be both scheduled and unscheduled. It is important to understand whether subject visits will be scheduled or unscheduled, completed in-person or remotely, will be single or repeat visits, and whether they will be part of a larger repeating visit cycle. As with every clinical trial, visits are individually defined. At each visit, a defined set of data is collected. A visit schedule is required when certain information must be collected on a timeline. For instance, a subject may be enrolled at Day 0 and then scheduled for treatment visits every seven (7) days for the first month, and then scheduled for follow-up visits every thirty (30) days for the next six (6) months. This schedule is applied to every subject on the trial to maintain consistency. Visit cycles and the cycle schedules consist of a collection of visits that are meant to be repeated over a specified duration. For instance, a cycle might consist of a dosing visit, a survey, labs, and a follow-up visit. This cycle may need to be repeated multiple times to collect an accurate response to treatment. Once the visits have been entered, this visual map defines which visits the subjects within the different groups are supposed to attend. Not all subjects attend all defined visits. Further, in an embodiment, a plurality of visits mapped against a plurality of subjects are to attend in accordance with the clinical trial design and clinical trial timeline generated. In contrast to a visit schedule which details the correlation between subjects and visits, the addition of a visual visit timeline details the scheduled dates and windows of the visits to which each subject is individually expected to participate based on their respective study enrollment date.

As with every clinical trial, forms are individually defined. Thus, the form definition requirement is directed to forms which are data sets that consist of many different data points that must be collected for analysis. Data points are grouped into like-minded data sets dictated by the FDA. For instance, all adverse event related data points would reside on an Adverse Event form. Additionally, nothing unrelated should be on this form. Once the forms have been entered, the form visit schedule requirement is directed to a visual map that defines which forms are collected at which visits. Not all forms are collected at all defined visits. Groups are not considered in this map. If anyone attends a defined visit, they must complete all forms assigned to this visit. Advantageously, in accordance with the principles of the disclosed embodiments, this is executed through a visual manager which is another feature not found in conventional EDC systems.

The data point definitions requirement addresses the requirement that data points must be individually defined for every clinical trial. For instance, a lab value data point would be limited to a numeric value. A single-select data point would be limited to one of a predefined list of selectable values. Data points must be uniquely identified across the entire clinical trial. Additionally, they must be identified with an export variable name and a CDISC-compliant variable name. Data points are further defined as a “data type” and a “display as”, each with unique properties. In the case of a “single-select” or “multi-select” data type, a code list must be defined with a list of potential selections. In this scenario, a code list is defined once with the ability to be used repeatedly in conjunction with multiple data points. The form visibility schedule requirement addresses the desire to have forms scheduled to enable or appear if certain data has been entered. For instance, a Pregnancy form should only appear if the subject is female and has childbearing potential. Another example is that it is prudent to only have Visit 2 appear after Visit 1 has been completed. This significantly reduces confusion and data collection mistakes. This is referred to as form visibility or “form rules”. Advantageously, in accordance with the principles of the disclosed embodiments, this is executed through a visual manager which is another feature not found in conventional EDC systems and a technical improvement thereover. Further, given it is essential to restrict access to system functionality, read/write capabilities, and access to specific forms and data the form accessibility schedule requirement requires user roles with specific permissions be created and users are assigned one or more user roles. All EDC software is required to have this, but none have a visual manager. Advantageously, in accordance with the principles of the disclosed embodiments, this is executed through a visual manager which is another feature not found in conventional EDC systems and is another technical improvement of the subject disclosure.

The advanced programming requirement addresses the notion that advanced programming is required in every system to create edit checks or queries to ensure that the data is entered correctly, logically makes sense, and is not in violation of the rules of the protocol. Typically, this is where designers and builders experience the most difficulty resulting in significant cost inflation for every trial using conventional clinical trial design techniques. As such, the disclosed embodiments herein provide for a feature connected to the Data Points requirement to simplify the creation of edit checks and queries which is accessible through both the Trial Requirements and Data Collection Specifications workflows. Further, a custom form rule builder is provided that is connected to the Form Visibility requirement to simplify the creation of the Form Rules.

Turning our attention again back to FIG. 4, having identified and specified the various clinical trial requirements this facilitates the automated generation of the clinical trial protocol (see, block 420), the informed consents (see, block 470), the CRFs (see, block 425) and the EDC specifications (see, block 430). Again, these are significant contributions and features made by the disclosed embodiments representing significant advance over conventional clinical trial designer systems, EDC system and techniques. At block 435 and starting a build phase 460, the trial design and clinical trial timeline generated, the clinical trial protocol generated, the informed consents generated, the CRFs generated, and the specifications generated are transmitted to an EDC system. Any existing EDC system may be utilized such that a clinical trial database design is returned by the EDC system that is specific to collecting data for the targeted clinical trial in accordance with the trial design and clinical trial timeline generated, the clinical trial protocol generated, the informed consents generated, the CRFs generated, and the EDC specifications generated thereby concluding the build phase 460. The returned clinical trial database design is then validated at block 440 to ensure the overall clinical design build and specifications match. Then, at block 445 and starting a live phase 465 live data collection proceeds in accordance with the clinical trial (as approved by the FDA) and is illustratively managed by the EDC system which also provides analysis at block 450 that includes various reports, exports, and visualizations.

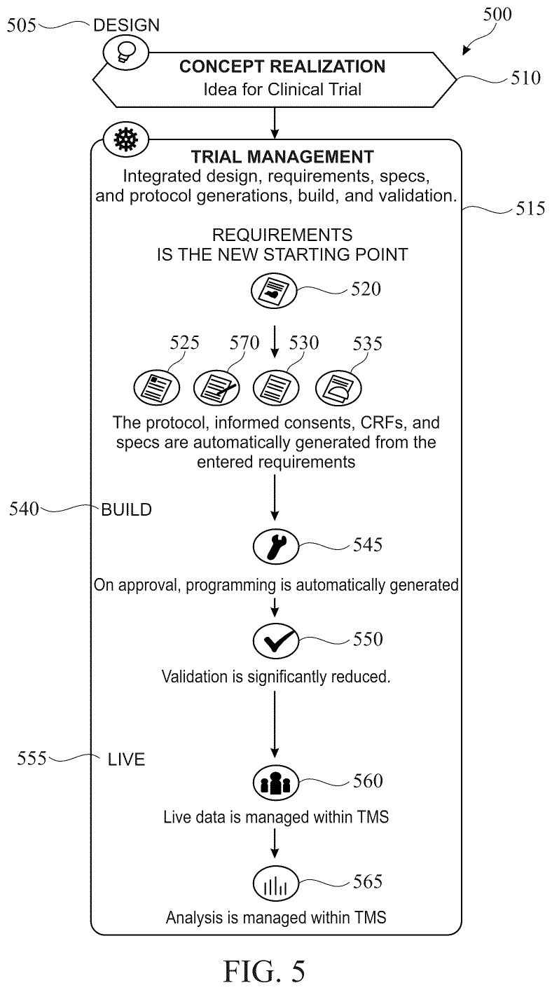

Turning our attention to FIG. 5, a holistic clinical trial design and management process architecture and workflow 500 that eliminates the need for an EDC system in accordance with an embodiment. As noted above, this is a further embodiment whereby the system and method are configured to completely eliminate the need and use of an EDC system in clinical design and management using the trial management system (TMS) architecture 515. The design phase 505 conforms exactly with the design phase 455 as detailed above such that blocks 520, 525, 530, 535, and 570 conform with the detailed description above with respect to blocks 410-430 and 470 of FIG. 4. However, in the subject embodiment, the build phase 540 incorporates programming directly such that a clinical trial database design is returned directly which is specific to collecting data for the targeted clinical trial in accordance with the trial design and clinical trial timeline generated, the clinical trial protocol generated at block 545, the informed consents generated, the CRFs generated, and the EDC specifications generated. Thus, at block 550 validation is conducted but given the vertical integration of the subject embodiment the time necessary to complete is significantly reduced. Then, at block 560 and starting a live phase 555 live data collection proceeds in accordance with the clinical trial (as approved by the FDA) and is illustratively vertically managed by the trial management system (again, no EDC system needed) which also provides analysis at block 565 that includes various reports, exports, and visualizations (again, no EDC system needed).

Turning our attention to FIG. 6, illustrative architectures for a trial designer app 600 and a trial design and management app 1000 are shown, respectively, in accordance with an embodiment. Each app is configured for delivering and executing the clinical trial design and management operations as discussed throughout this disclosure. As will be appreciated, the architectures may be used, illustratively, in conjunction with the trial designer and management system 800, the user device 900 or any other data processing system for launching and executing the trial designer app 600 and/or the trial designer and management app 1000 and their associated operations. As shown, the architecture for the operations of the trial designer app 600 and the trial designer and management app 1000 provides several interfaces and engines used to perform a variety of functions such as the collection, aggregation, manipulation, processing, analyzing, verification, authentication, and display of applicable real-time information and data that are useful to realize the delivery of the operations for clinical trial design and management in accordance with the disclosed embodiments. More particularly, data display interface module 618 and communications module 612 are used to facilitate the input/output and display of electronic data and other information to, illustratively, the users employing the trial designer and management system 800 (e.g., a touch screen of the trial designer and management system 800) and executing the trial designer app 600 and/or the trial designer and management app 1000. The data collection module 606 facilitates data gathering from various users, clinical trial subjects/participants, and other third parties. The communications module 612 may include a location-based services module that provides for the delivery of location-based services in order for the geographic locations of the users to be identified and displayed (e.g., GPS locations). The communications module 612 will also facilitate communications by and through the trial designer and management system 800, for example.

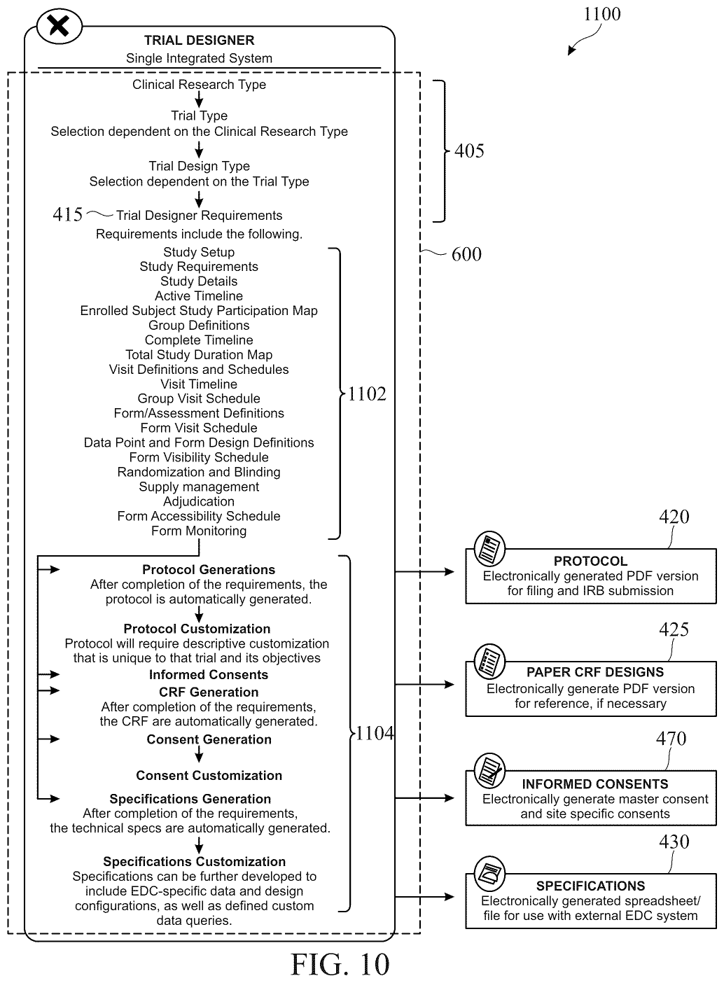

Execution engine 602 may be employed to deliver the operations for clinical trial design and management through the execution of the trial designer app 600 and/or the trial designer and management app 1000. In such delivery, the execution engine 602 will operate and execute with at least the following program modules: trial requirements module 604, data collection module 606, protocol writer module 608, consent builder module 610, communications module 612, trial designer operations module 614, design sign-off module 616, data display interface module 618, custom programming module 620, validation module 622, build sign-off module 624, deployment administration and management module 626, CRF module 628, specification module 630, group definitions module 632, total study duration map module 634, active timeline module 636, visits administration and management module 638, group schedule module 640, and forms and data module 642 (which may also include an audit trail module for tracking the name of the user and the time stamp for every piece of clinical data that is either entered or changed with the live version only). The trial designer and management app 1000 further comprises programming module 644, live data collection module 646, analysis module 648, and trial management system operations module 650. Further, in an embodiment, data display interface module 618 may include a graphical user interface (GUI) module, and the communications module 612 are used to facilitate the input/output and display of electronic data and other information (e.g., a GUI) to, illustratively, the users employing the trial designer and management system 800 (e.g., a touch screen) and executing the trial designer app 600 and/or the trial designer and management app 1000. The data collection module 606 facilitates various types of data collection from the various users and systems (e.g., the EDS system) involved in the clinical trial design and management operations of the disclosed embodiments. Again, the operations executed by each and every of the foregoing modules are, for example, as discussed throughout this disclosure. For example, and turning our attention briefly to FIG. 10, a high-level overlay diagram 1100 of the trial design and workflow architecture of FIG. 4 with the illustrative architectures for the trial designer app of FIG. 6 in accordance with an embodiment. More particularly, consistent with the details above, the trial designer app 600 will facilitate clinical trial design and management using an automated and integrated architecture that utilizes a clinical trial idea and concept realization (see also block 405 in FIG. 4) which includes selecting and identifying a clinical research type, a clinical trial type (dependent upon the clinical research type), and a trial design type (dependent upon the clinical trial type). This transitions to identifying and defining specific requirements 1102 (see also block 415 in FIG. 4) for the automated generation 1104 of the protocol 420, the informed consents 470, the CRFs 425, and the specifications 430 (see also FIG. 4).

Those skilled in the art will appreciate that the present disclosure contemplates the use of systems configurations and/or computer instructions that may perform any or all of the operations involved in the predictive modelling using segmental motion and tracking operations herein. The disclosure of computer instructions that include, for example, the trial designer app 600, the trial designer and management app 1000, and the trial designer and management system 800 is not meant to be limiting in any way. Those skilled in the art will readily appreciate that stored computer instructions and/or systems configurations may be configured in any way while still accomplishing the various goals, features, and advantages according to the present disclosure. The terms “program,” “application,” “software application,” and the like as used herein, are defined as a sequence of instructions designed for execution on a computer system. A “program,” “computer program,” “application,” or “software application” may include a subroutine, a function, a procedure, an object method, an object implementation, an executable application, an applet, a servlet, a source code, an object code, a shared library/dynamic load library, and/or other sequence of instructions designed for execution on a computer system. Accordingly, the applications herein may be written using any number of programming languages and/or executed on compatible platforms that will be well understood by those skilled in the art. Computer readable program instructions for carrying out operations of the disclosed embodiments may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on one or more standalone computers, partly on one or more standalone computers, as a stand-alone software package, partly on one or more standalone computers and partly on one or more remote computers, partly on one or more standalone computers and partly on one or more distributed computing environments (such as a cloud environment), partly on one or more remote computers and partly on one or more distributed computing environments, entirely on one or more remote computers or servers, or entirely on one or more distributed computing environments. Standalone computers, remote computers, and distributed computing environments may be connected to each other through any type of network or combination of networks, including local area networks (LANs), wide area networks (WANs), through the Internet (e.g., using an Internet Service Provider), or the connection may be made to external computers.

Turning our attention to FIG. 7, a high-level block diagram of a cloud network services architecture 700 for providing a trial designer system that facilitates clinical trial design and management using an automated and integrated architecture in accordance with an embodiment. As shown for instance in FIG. 7, the cloud network services architecture 700 includes a cloud 702 comprising at least server(s) 704, access point(s) 706 and database(s) 708. As will be detailed herein below, the cloud 702 facilitates the delivery of the online clinical trial design and management services using trial designer and management system 800 to a plurality of users (e.g., the plurality users comprised by clinical trial client 1 710-1 through clinical trial client N 710-N, functional service provider (FSP) 1 718-1 through FSP N 718-N). In an embodiment, the online clinical trial design and management services processing, offered by and through the cloud network services architecture 700 and trial designer and management system 800 will be facilitated by a trial designer app 600 (see, FIG. 4), as will be detailed herein below, executing on a user device 900 (see, FIG. 3). The user device 900 provides the various users (e.g., clinical trial client 1 710-1, clinical trial client N 710-N, FSP 1 718-1, and/or FSP N 718-N) with real-time access to online clinical trial design and management services in accordance with the disclosed embodiments herein.

As noted above, the cloud 702 comprises at least server(s) 704, the access point(s) 706 and the database(s) 708. Cloud, cloud service, cloud server and cloud database are broad terms and are to be given their ordinary and customary meaning to one of ordinary skill in the art and includes, without limitation, any database, data repository or storage media which store content typically associated with and managed by clinical trial users, clinical trial participants, EDC system 738, governmental agencies (e.g., FDA and other governmental agencies 720) and third-party resource providers (e.g., third-party resource providers 714) in the context of online clinical trial design and management services, to name just a few. A cloud service may include one or more cloud servers and cloud databases that provides for the remote storage of content as hosted by a third-party service provider or operator. A cloud server may include an HTTP/HTTPS server sending and receiving messages in order to provide web-browsing interfaces to client web browsers as well as web services to send data to integrate with other interfaces (e.g., as executed on the user device 900). The cloud server may be implemented in one or more servers and may send and receive content in a various forms and formats, user supplied and/or created information/content and profile/configuration data that may be transferred from or stored in a cloud database (e.g., the databases 708). A cloud database may include one or more physical servers, databases or storage devices as dictated by the cloud service's storage requirements. The cloud database may further include one or more well-known databases (e.g., an SQL database) or a fixed content storage system to store content, user profile information, configuration information, administration information and any other information necessary to execute the cloud service. In various embodiments, one or more networks providing computing infrastructure on behalf of one or more users may be referred to as a cloud, and resources may include, without limitation, data center resources, applications (e.g., software-as-a-service or platform-as-a-service) and management tools.

Turning our attention to FIG. 8, an illustrative configuration for the trial designer and management system 800 is shown for deployment in the cloud network services architecture 700 and providing for clinical trial design and management in accordance with the disclosed embodiments. As shown, the trial designer and management system 800 comprises processor 802 for executing program code (e.g., trial designer app 600) and communications interface 814 for managing communications to and from the trial designer and management system 800, memory 806 and/or read-only memory (ROM) 808 for storing program code and data, and power source 818 for powering the trial designer and management system 800. The memory 806 is coupled to the bus 804 for storing computer-readable instructions to be executed by the processor 802 (e.g., execution of the trial designer app 600). Database manager 812 is used to manage the delivery and storage of content, data, and other information in the trial designer system database(s) 724, database(s) 708 and across third-party resource providers, for example, as shown in FIG. 7. The trial designer system database(s) 724 may store and provide information including, but not limited to, forms, reports and analysis 726, trial requirements 728, protocols 730, specifications 732, and clinical trials and data 734. As will be detailed further herein below, the operations performed by the trial designer and management system 800 and/or the user device(s) 900 in combination with the trial designer app and/or trial management system app 1000 provide for clinical trial design and management in accordance with the principles of the disclosed embodiments.

Website manager 820 is used to deliver and manage content, data, and other information across one or more websites that may be utilized to access and use the trial designer and management system 800, for example. Further, the operations provided by and through the trial designer app 600 and/or the trial management system app 1000 may be offered through a web-based application. As will be discussed herein, the trial designer app 600 and/or the trial management system app 1000, as stored in data storage 810, when executed by the processor 802 will enable access by a plurality of parties (e.g., clinical trial client 1 710-1 through clinical trial client N 710-N and/or FSP 1 718-1 through FSP N 718-N) to the trial designer and management system 800 for the processing of, for example, the forms, reports and analysis 726, trial requirements 728, protocols 730, specifications 732, and clinical trials and data 734. Location-based service manager 822 facilitates the delivery of location-based services (e.g., GPS tracking) either independently or on user device 900 thereby allowing the trial designer and management system 800 to register the exact location of the user of the user device 900, for example, as the clinical trial client 1 710-1 roams from one location to another location such that the clinical trial design and management processing hereunder may be tailored to a current location and/or the needs of the user may change based on their current location.

In an embodiment, the clinical trial design and management processing provided through the execution of the trial designer app 600 and/or the trial management app 1000 may also include a web-based delivery platform and/or accessing and interfacing any number of web using website manager 820 for procuring information and data that can be used in the trial designer and management system 800. The term “website” in the context herein is used in a conventional and broadest sense and is located on at least one server containing web pages stored thereon and is operational in a 24-hour/7-day typical fashion. Further, as shown in the cloud network services architecture 700, the plurality of parties (i.e., clinical trial client 1 710-1 through clinical trial client N 710-N and/or FSP 1 718-1 through FSP N 718-N) may alternatively utilize well-known Internet 722 for access to trial designer and management system 800 by and through a web browser on the user device 900, for example. The trial designer and management system 800 may also include one or more input/output devices 816 that enable user interaction with the trial designer and management system 800 (e.g., camera, display, keyboard, mouse, speakers, microphone, buttons, etc.). The input/output devices 816 may include peripherals, such as a camera, printer, scanner, touchscreen display, etc. For example, the input/output devices 816 may include a display device such as a cathode ray tube (CRT), plasma monitor, liquid crystal display (LCD) monitor or organic light-emitting diode (OLED) monitor for displaying information to the user, a keyboard, and a pointing device such as a mouse or a trackball by which the user can provide input to the trial designer and management system 800 or an associated display device 828, for example, that may also be managed by graphical user interface 824.

The communications interface 814 is used to facilitate communications across the communications links 736 (see, FIG. 7) within the cloud network services architecture 700. This may take the form, for example, of a wide area network connection that communicatively couples the trial designer and management system 800 with the access points 706 which may be a cellular communications service. Similarly, communications managed by the communications interface 814 may take the form, for example, of a local Wi-Fi network interface or Ethernet interface the communicatively couples the trial designer and management system 800 with the Internet 722, local area network (LAN) 712 and ultimately the trial designer and management system 800 and/or the user device 900. In the instant embodiment, the trial designer app 600 (or the trial management system app 1000) and/or the communications interface 814 may include a communications stack for facilitating communications over the respective communications link 736. Electronic communications by and through trial designer and management system 800 between the various systems, networks, devices, users, entities, and/or individuals are facilitated by the communications links 736 in accordance with any number of well-known communications protocols and methods (e.g., wireless communications).

Turning our attention briefly to FIG. 9, an illustrative user device 900 is shown configured in accordance with an embodiment. The user device 900 typically includes bus 902 and processor 904 coupled to the bus 902 for executing operations and processing information. As will be appreciated, a “user device” in the context herein may comprise a wide variety of devices such as any type of hardware device, mobile devices, smartphones, laptop computers, desktop computers, kiosks, tablets, and wearable device, to name just a few, that execute applications (e.g., a mobile application) in accordance with the principles of the disclosed embodiments herein. The processor 904, as powered by power source 912, may include both general and special purpose microprocessors, and may be the sole processor or one of multiple processors of the device. This is equally applicable to the processor 802 of FIG. 8. Further, the processor 904 (or the processor 802) may comprise one or more central processing units (CPUs) and may include, be supplemented by, or incorporated in, one or more application-specific integrated circuits (ASICs) and/or one or more field programmable gate arrays (FPGAs).

The user device 900 may also include memory 906 coupled to the bus 902 for storing computer-readable instructions to be executed by the processor 904. The memory 906 may also be utilized for storing temporary variables or other intermediate information during the execution of the instructions by the processor 904. The user device 900 may also include ROM 908 or other static storage device coupled to the bus 902. Further, data storage device 910, such as a magnetic, optical, or solid-state device may be coupled to the bus 902 for storing information and instructions for the processor 904 including, but not limited to, the trial designer app 600 and/or the trial management system app 1000. Data storage device 910 (or the data storage device 810) and the memory 906 (and the memory 906) may each comprise a non-transitory computer readable storage medium and may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

The user device 900 may also include one or more communications interface 916 for communicating with other devices via a network (e.g., a wireless communications network) or communications protocol (e.g., Bluetooth®). For example, such communication interfaces may be a receiver, transceiver, or modem for exchanging wired or wireless communications in any number of well-known fashions. For example, the communications interface 916 (or the communications interface 814) may be an integrated services digital network (ISDN) card or modem/router used to facilitate data communications of various well-known types and formats. Further, illustratively, the communications interface 916 (or the communications interface 814) may be a LAN card used to provide data communication connectivity to a comparable LAN. Wireless communication links may also be implemented. The Global Positioning System (GPS) transceiver 918 and antenna 920 facilitate delivery of location-based services in order to register the exact location of the user device 900, for example, as the user roams from one location to another location. As will be understood, the application herein will be able to track individual users and their location upon the launching of the application thereby enabling the well understood GPS location features of the user device 900 (e.g., a smartphone).

As will be appreciated, the functionality of the communication interface 916 (or the communications interface 814) is to send and receive a variety of signals (e.g., electrical, optical, or other signals) that transmit data streams representing various data types. The user device 900 may also include one or more input/output devices 914 that enable user interaction with the user device 900 (e.g., camera, display, keyboard, mouse, speakers, microphone, buttons, etc.). The input/output devices 914 (or I/O devices 816) may include peripherals, such as a camera, printer, scanner, touchscreen display, etc. For example, the input/output devices 914 (or the I/O devices 816) may include a display device such as a cathode ray tube (CRT), plasma monitor, liquid crystal display (LCD) monitor or organic light-emitting diode (OLED) monitor for displaying information to the user, a keyboard, and a pointing device such as a mouse or a trackball by which the user can provide input to the user device 900 or an associated display device, for example.

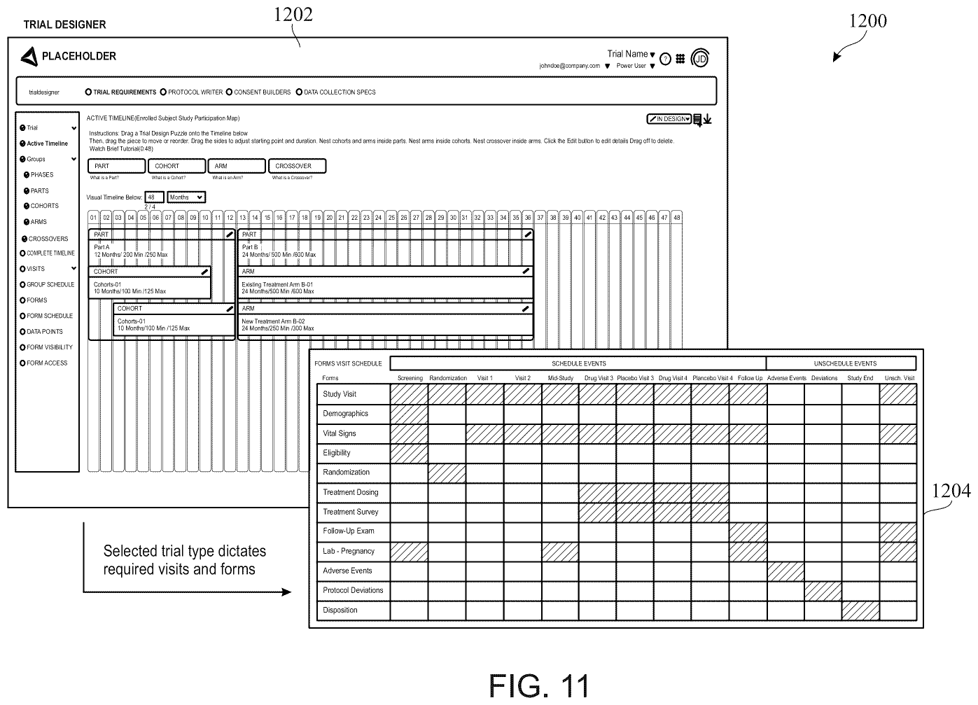

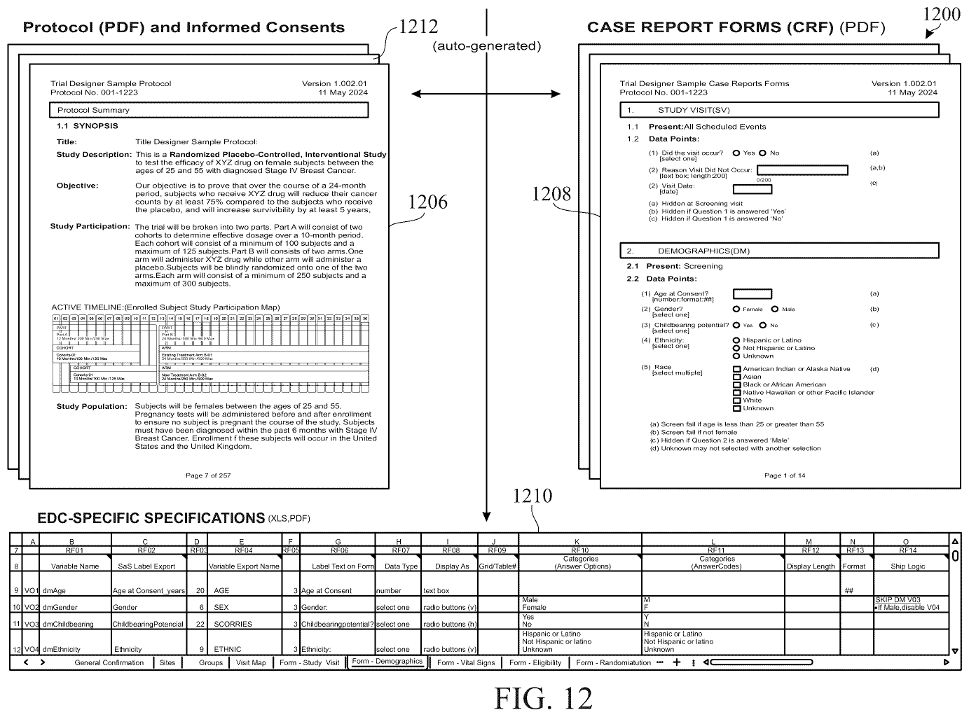

Turning our attention to FIGS. 11 and 12, illustrative outputs from executing the trial design app of FIG. 6 employing the trial design and workflow architecture of FIG. 10 in accordance with an embodiment. More particularly, GUI 1200 which may appear on the displays of the trial designer and management system 800 and/or the user device 900, for example, shows automatically generated output (e.g., from executing the trial designer app 600 as detailed hereinabove) comprising active timeline 1202, form visit schedule 1204, protocol 1206, informed consents 1212, CRFs 1208, and EDC-specific specifications 1210. Such automated generation, compilation, and display is another advantage over conventional clinical trial design techniques and EDC systems.

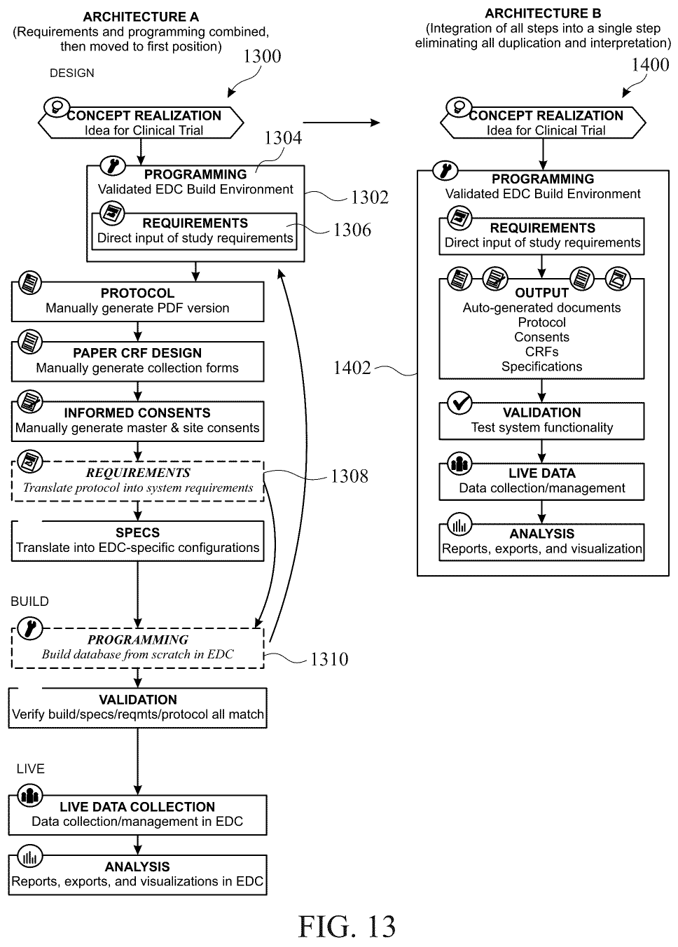

As detailed above, in accordance with the principles of the disclosed embodiments, the typical complex inner workings of clinical trial design are simplified and automated for providing improved clinical design and ensuring FDA and CDISC compliance, and dramatically reducing development timelines and costs. Significantly, the principles of the disclosed embodiments starts the clinical design process by identifying and gathering clinical trial requirements which is in contrast to conventional clinical trial design processes that begin with a manually developed protocol. In this way, the system and method herein use the identified and gathered clinical trial requirements for automatically generating a clinical trial design and clinical trial timeline, a clinical trial protocol, one or more informed consents, one or more CRFs, and one or more EDC specifications. All this is generated while simultaneously assembling designs pre-populated with a standard trial architecture and required data points, validating configuration, cross-checking enrollment numbers and study timelines, populating the protocol with supporting data and visuals, and generating a customized technical specifications document for use with an existing clinical EDC system. In a further embodiment, the system and method are configured to completely eliminate the need and use of an EDC system in clinical design and management. To further illustrate the advantages of these principles, FIG. 13 presents a high-level visualization of the clinical trial design process architecture and workflows herein as compared to traditional systems in accordance with an embodiment. More particularly, the clinical trial design process architecture and workflow of architecture A 1300 moves and integrates the traditional requirements aspect (see block 1308 showing its traditional position in the workflow) and programming aspect (see block 1310 showing its traditional position in the workflow) aspects as shown in block 1302 comprises of both programming 1302 and requirements 1306 in accordance with the principles of the disclosed embodiments. Architecture A is then condensed into architecture B 1400 which presents a holistic clinical trial design and management process and workflow 1402 that eliminates the need for any EDC system in clinical design and management. See FIG. 5 and the discussion herein above for details which will not be repeated here with respect to this EDC elimination architecture.

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention. A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices. “Software” may refer to prescribed rules to operate a computer. Examples of software may include code segments in one or more computer-readable languages; graphical and/or textual instructions; applets; pre-compiled code; interpreted code; compiled code; and computer programs. A network is a collection of links and nodes (e.g., multiple computers and/or other devices connected together) arranged so that information may be passed from one part of the network to another over multiple links and through various nodes. Examples of networks include the Internet, the public switched telephone network, wireless communications networks, computer networks (e.g., an intranet, an extranet, a local-area network, or a wide-area network), wired networks, and wireless networks.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block dia-grams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media. When a single device or article is described herein, it will be readily apparent that more than one device/article (whether or not they cooperate) may be used in place of single device/article. Similarly, where more than one device or article is described herein (whether or not they cooperate), it will be readily apparent that a single device/article may be used in place of the more than one device or article. The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-transitory, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, a RAM, a PROM, an EPROM, a FLASH-EEPROM, removable media, flash memory, a “memory stick”, any other memory chip or cartridge, a carrier wave as described hereinafter, or any other medium from which a computer can read. Various forms of computer readable media may be involved in carrying sequences of instructions to a processor. For example, sequences of instruction may be delivered from RAM to a processor, may be carried over a wireless transmission medium, and/or may be formatted according to numerous formats, standards or protocols, such as Bluetooth, 4G, 5G, etc.

Where databases are described, it will be understood by one of ordinary skill in the art that alternative database structures to those described may be readily employed, and other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a non-transitory computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units. A “network” may refer to a number of computers and associated devices that may be connected by communication facilities. A network may involve permanent connections such as cables or temporary connections such as those made through the telephone or other communication links. A network may further include hard-wired connections (e.g., coaxial cable, twisted pair, optical fiber, waveguides, etc.) and/or wireless connections (e.g., radio frequency waveforms, free space optical waveforms, acoustic waveforms, etc.). Examples of a network may include: an internet, such as the Internet; an intranet; a LAN; a wide area network (WAN); and a combination of networks, such as an internet and an intranet.

As noted above, in some embodiments the method or methods described above may be executed or carried out by a computing system including a non-transitory computer-readable storage medium, also described herein as a storage machine, that holds machine-readable instructions executable by a logic machine (i.e., a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a GUI, or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard, or gaming controller. For example, a user input may indicate a request that certain task is to be executed by the computing system, such as requesting the computing system to display any of the above-described information or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Thus, the steps of the disclosed method(s) and the associated discussion herein above can be defined by the computer program instructions stored in a memory and/or data storage device and controlled by a processor executing the computer program instructions. Accordingly, by executing the computer program instructions, the processor executes an algorithm defined by the disclosed method. For example, the computer program instructions can be implemented as computer executable code programmed by one skilled in the art to perform the illustrative operations defined by the disclosed methods. Further, it will be appreciated that any flowcharts, flow diagrams, state transition diagrams, pseudo code, program code and the like represent various processes which may be substantially represented in computer readable medium and so executed by a computer, machine, or processor, whether or not such computer, machine or processor is explicitly shown. One skilled in the art will recognize that an implementation of an actual computer or computer system may have other structures and may contain other components as well, and that a high-level representation of some of the components of such a computer is for illustrative purposes.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A clinical trial design and management system comprising:
a processor;
a memory storing instructions that when executed cause the processor to perform operations comprising:
receiving a plurality of clinical trial requirements specific to a targeted clinical trial having a clinical research type and a trial type;
generating from the plurality of clinical trial requirements received (i) a clinical trial design and clinical trial timeline; (ii) a clinical trial protocol; (iii) one or more informed consents; (iv) one or more case report forms (CRFs); and (v) one or more electronic data capture (EDC) specifications;
transmitting the trial design and clinical trial timeline generated, the clinical trial protocol generated, the one or more informed consents generated, the one or more CRFs generated, and the one or more EDC specifications generated to an EDC system;
receiving from the EDC system a clinical trial database design specific to collecting data for the targeted clinical trial in accordance with the trial design and clinical trial timeline generated, the clinical trial protocol generated, the one or more informed consents generated, the one or more case report forms generated, and the one or more EDC specifications generated; and
validating the clinical trial database design received using the one or more EDC specifications generated.

2. The clinical trial design and management system of claim 1, wherein the operations performed by the processor further comprise:
generating one or more location-specific clinical research participant consent documents.

3. The clinical trial design and management system of claim 1, wherein the operations performed by the processor further comprise:
presenting the clinical trial database design validated for user approval; and
responsive to the presentation of the clinical trial database design validated, receiving the user approval.

4. The clinical trial design and management system of claim 1, wherein the operations performed by the processor further comprise:
recommending, based on the targeted clinical trial, the clinical research type and the trial type.

5. The clinical trial design and management system of claim 1, wherein the trial design and clinical trial timeline, the clinical trial protocol, the one or more informed consents, the one or more CRFs, and the one or more EDC specifications are generated using a common workflow.

6. The clinical trial design and management system of claim 1, wherein the operations performed by the processor further comprise:
correlating the clinical trial database design received, the trial design and clinical trial timeline generated, the clinical trial protocol generated, the one or more informed consents generated, the one or more CRFs generated, and the one or more EDC specifications generated with one or more standards promulgated by a Clinical Data Interchange Standards Consortium (CDISC).

7. The clinical trial design and management system of claim 1, wherein the operations performed by the processor further comprise:
accessing a library of common calculations associated with the targeted clinical trial.

8. The clinical trial design and management system of claim 7, wherein the operations performed by the processor further comprise:
nesting multiple ones of the common calculations from the library of common calculations in at least one case report from the one or more CRFs generated.

9. The clinical trial design and management system of claim 8, wherein the clinical trial design and clinical trial timeline generated provide for clinical trial data collection on a per site level and a per study level.

10. The clinical trial design and management system of claim 1, wherein the generating from the plurality of clinical trial requirements received (i) a clinical trial design and clinical trial timeline; (ii) a clinical trial protocol; (iii) one or more CRFs; and (iv) one or more EDC specifications further comprises:
defining one or more groups and one or more crossovers; and
incorporating the one or more groups and the one or more crossovers defined into at least the clinical trial timeline generated.

11. The clinical trial design and management system of claim 10, wherein the one or more groups defined further comprise a plurality of categories defined by phases, parts, cohorts, arms, and crossovers.

12. The clinical trial design and management system of claim 11, wherein each one of the phases, parts, cohorts, arms, and crossovers require a start date, duration, enrollment goal, and enrollment limit.

13. The clinical trial design and management system of claim 12, wherein the one or more crossovers defined further comprise a crossover type, related arms, crossover direction, at least two potential start dates, subject eligibility, and maximum number of subjects allowed to crossover.

14. The clinical trial design and management system of claim 10, wherein the operations performed by the processor further comprise:
mapping a plurality of visits for which a plurality of subjects are to attend in accordance with the clinical trial design and clinical trial timeline generated.

15. A clinical trial design and management system comprising:
a processor;
a memory storing instructions that when executed cause the processor to perform operations comprising:
receiving a plurality of clinical trial requirements specific to a targeted clinical trial;
recommending, based on the targeted clinical trial, a clinical research type and a trial type for the targeted clinical trial;
generating from the plurality of clinical trial requirements received (i) a clinical trial design and clinical trial timeline; (ii) a clinical trial protocol; (iii) one or more informed consents; (iv) one or more case report forms (CRFs); and (v) one or more electronic data capture (EDC) specifications;
generating one or more location-specific clinical research participant consent documents;
mapping a plurality of visits for which a plurality of subjects are to attend in accordance with the clinical trial design and clinical trial timeline generated.
transmitting the trial design and clinical trial timeline generated, the clinical trial protocol generated, the one or more informed consents, the one or more CRFs generated, and the one or more EDC specifications generated to an EDC system;
receiving from the EDC system a clinical trial database design specific to collecting data for the targeted clinical trial in accordance with the trial design and clinical trial timeline generated, the one or more informed consents generated, the clinical trial protocol generated, the one or more CRFs generated, and the one or more EDC specifications generated; and
validating the clinical trial database design received using the one or more EDC specifications generated.

16. The clinical trial design and management system of claim 15, wherein the generating from the plurality of clinical trial requirements received (i) a clinical trial design and clinical trial timeline; (ii) a clinical trial protocol; (iii) one or more CRFs; and (iv) one or more EDC specifications further comprises:
defining one or more groups and one or more crossovers, wherein the one or more groups defined further comprise a plurality of categories defined by phases, parts, cohorts, arms, and crossovers; and
incorporating the one or more groups and the one or more crossovers defined into at least the clinical trial timeline generated.

17. The clinical trial design and management system of claim 16, wherein each one of the phases, parts, cohorts, arms, and crossovers require a start date, duration, enrollment goal, and enrollment limit.

18. A non-transitory computer medium having executable code stored thereon, that when executed, cause a data processing system to perform operations comprising:
receiving a plurality of clinical trial requirements specific to a targeted clinical trial having a clinical research type and a trial type;
generating from the plurality of clinical trial requirements received (i) a clinical trial design and clinical trial timeline; (ii) a clinical trial protocol; (iii) one or more informed consents; (iv) one or more case report forms (CRFs); and (v) one or more electronic data capture (EDC) specifications;
transmitting the trial design and clinical trial timeline generated, the clinical trial protocol generated, the one or more informed consents generated, the one or more CRFs generated, and the one or more electronic data capture (EDC) specifications generated to an EDC system;
receiving from the EDC system a clinical trial database design specific to collecting data for the targeted clinical trial in accordance with the trial design and clinical trial timeline generated, the one or more informed consents generated, the clinical trial protocol generated, the one or more CRFs generated, and the one or more EDC specifications generated; and
validating the clinical trial database design received using the one or more EDC specifications generated.

19. The non-transitory computer medium of claim 18, wherein the operations performed by the data processing system further comprise:
defining one or more groups and one or more crossovers, wherein the one or more groups defined further comprise a plurality of categories defined by phases, parts, cohorts, arms, and crossovers, and the one or more crossovers defined further comprise a crossover type, related arms, crossover direction, at least two potential start dates, subject eligibility, and maximum number of subjects allowed to crossover; and
incorporating the one or more groups and the one or more crossovers defined into at least the clinical trial timeline generated.

20. The non-transitory computer medium of claim 18, wherein the operations performed by the data processing system further comprise:
mapping a plurality of visits for which a plurality of subjects are to attend in accordance with the clinical trial design and clinical trial timeline generated.
